(19) United States
(12) Patent Application Publication
(10) Pub. No.: US 2015/0278439 A1
(43) Pub. Date: Oct. 1, 2015

(54) METHOD FOR QUICK-SEARCH OF LOCI-OF-INTEREST IN A GENE SEQUENCE OF A TARGET BIOLOGICAL VIRUS

(71) Applicant: Chaoyang University of Technology, Taichung City (TW)

(72) Inventors: Wen-Goang Yang, Taichung (TW); Shulhn-Der Wang, Taichung (TW); Yu-Jen Hu, Taichung (TW); Kuan-Chih Chow, Taichung (TW)

(21) Appl. No.: 14/482,506

(22) Filed: Sep. 10, 2014

(30) Foreign Application Priority Data
Mar. 28, 2014 (TW) 103111788

(51) Int. Cl. G06F 19/22; G06F 19/14; C12Q 1/70

(52) U.S. Cl. CPC G06F 19/22; C12Q 1/70; G06F 19/14

(57) ABSTRACT

In a method for quick-search of loci-of-interest in a gene sequence of a target biological virus, a computer executes a phylogenetic algorithm to generate phylogenetic tree information, which is generated based on a selected gene segment of the target biological virus and a corresponding gene segment of each of related biological viruses. A set of to-be-matched biological viruses is determined based on the phylogenetic tree information. A computer matches the gene sequences of the to-be-matched biological viruses in the set so as to find the loci-of-interest in the gene sequence of the target biological virus.

### Cross-Reference to Related Applications

This application claims the benefit of Taiwanese Application No. 103111788 filed on Mar. 28, 2014. The content of which is hereby incorporated by reference in its entirety.

### BACKGROUND OF THE INVENTION

1. Field of the Invention

The present invention relates to a method for quick-search of loci-of-interest in a gene sequence of a target biological virus.

2. Background Information

In vaccine research and development, the process of finding viral genomic loci that can be used for developing vaccines is like buying a lottery ticket, of which the winning probability is little. Presently, the World Health Organization (WHO) has promulgated several experimental procedures, as well as pre-clinical and clinical operations. However, researchers are unable to determine which loci in the viral genes are critical for predicting biological virus mutations (such loci are usually related to antigen binding specificity). Therefore, a more advanced stratagem that combines biotechnological methods, including bioinformatics, statistics, mathematics, immunology and molecular biology, is essential for predicting virus mutation sites, which may affect the efficacy of the future vaccine program. Mostly, the vaccine developed will cause no severe side-effect and no harm to the general population. In addition, such rendition is able to reduce both time and costs in the development of vaccines.

### SUMMARY OF THE INVENTION

According to the present invention, a computer-implemented method is provided to quick-search for loci-of-interest in a gene sequence of a target biological virus. The method comprises:

A) finding a set of to-be-matched biological viruses from a group of related biological viruses that are related to the target biological virus, including
a1) generating phylogenetic tree information using at least one computer that executes at least one phylogenetic algorithm, the phylogenetic tree information being generated based on at least one selected gene segment of the gene sequence of the target biological virus and a corresponding at least one gene segment of a gene sequence of each of the related biological viruses in the group, the corresponding at least one gene segment corresponding to the at least one selected gene segment, and
a2) determining the set of to-be-matched biological viruses based on the phylogenetic tree information thus generated; and

B) matching, using a computer, the gene sequences of the to-be-matched biological viruses in the set so as to find the loci-of-interest in the gene sequence of the target biological virus.

### BRIEF DESCRIPTION OF THE DRAWINGS

Other features and advantages of the present invention will become apparent in the following detailed description of the embodiment with reference to the accompanying drawings, of which:

FIG. 1 is a flow chart of a computer-implemented method for quick-search of loci-of-interest in a gene sequence of a target biological virus according to an embodiment of the present invention;

FIG. 2 is a tree diagram illustrating a first phylogenetic tree in the embodiment of the present invention; and

FIG. 3 is a tree diagram illustrating a second phylogenetic tree in the embodiment of the present invention.

### DETAILED DESCRIPTION OF AN ILLUSTRATIVE EMBODIMENT

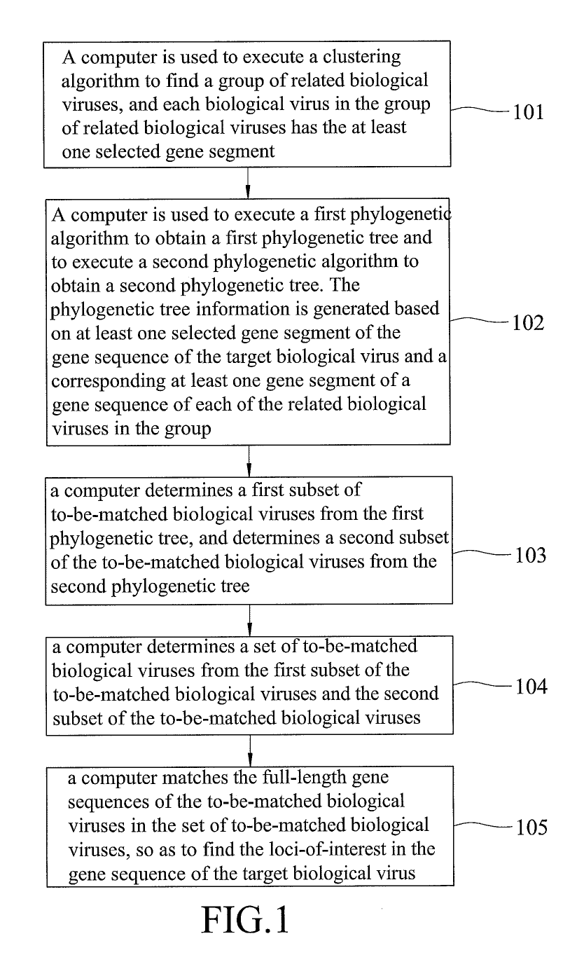

FIG. 1 illustrates an embodiment of a computer-implemented method for quick-search of loci-of-interest in a gene sequence of a target biological virus according to the present invention. In this embodiment, the target biological virus is exemplified using the H7N9 influenza A virus, which includes eleven gene segments, namely PB2, PB2-F1, PB1, PA, HA, NP, NA, M1, M2, NS1 and NS2. In this embodiment, only PB2 gene segment, NA (neuraminidase) gene segment and HA (hemagglutinin) gene segment will be mentioned hereinafter.

The term “loci-of-interest” as used herein refers to possible immune genomic loci that may involve immunogenicity and that may be included in a gene sequence that encodes, e.g., epitope-bearing peptides/protein. The “loci-of-interest” are predicted to be easily mutated and are associated with mutation loci among gene sequences of related biological viruses.

The embodiment of the computer-implemented method for quick-search of loci-of-interest in a gene sequence of a target biological virus includes the following steps 101 to 105.

In step 101, a computer is used to execute a clustering algorithm to find a group of related biological viruses from a genus of a family of biological viruses to which the target biological virus belongs. The clustering algorithm operates based on at least one selected gene segment of the gene sequence of the target biological virus (H7N9 in this embodiment) and a corresponding at least one gene segment of a gene sequence of each of the biological viruses in the genus (i.e., each biological virus in the group of related biological viruses has the at least one selected gene segment). In this embodiment, gene sequences of 170 influenza A viruses were retrieved from the National Center for Biotechnology Information (NCBI) database. Both the selected gene segment of the gene sequence of the targeted H7N9 biological virus and the corresponding gene segment of the gene sequence of each of the other influenza A viruses operated upon by the clustering algorithm are PB2 gene segment that encodes PB2 RNA polymerase, such that the group of related biological viruses from the 170 influenza A viruses to which H7N9 belongs is found. In this embodiment, the clustering algorithm is the Unweighted Pair Group Method with Arithmetic Mean (UPGMA) algorithm, and after executing the clustering algorithm, a group of thirteen, related biological viruses from the influenza A virus family to which H7N9 belongs is obtained. The group of thirteen related biological viruses includes H1N1, H1N2, H2N2, H3N2, H3N8, H5N1, H5N2, H5N3, H7N1, H7N2, H7N3, H7N7 and H10N7.

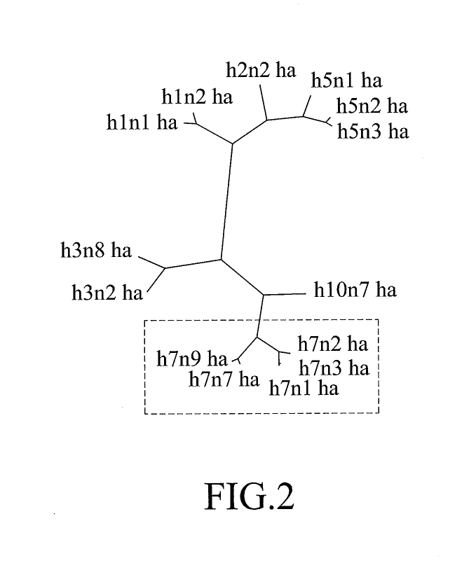

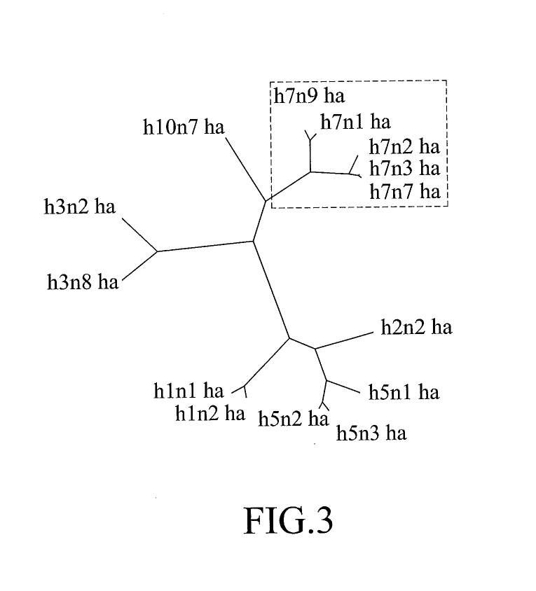

In step 102, a computer is used to execute a first phylogenetic algorithm to obtain a first result (a first phylogenetic tree in FIG. 2) and to execute a second phylogenetic algorithm to obtain a second result (a second phylogenetic tree in FIG. 3). The phylogenetic tree information is generated based on at least one selected gene segment of the gene sequence of the target biological virus (i.e., H7N9) and a corresponding at least one gene segment (corresponding to the at least one selected gene segment) of a gene sequence of each of the related biological viruses in the group. In this embodiment, the first phylogenetic algorithm is the Unweighted Pair Group Method with Arithmetic Mean (UPGMA) algorithm of the Clustalx computer program, and the second phylogenetic algorithm is the maximum-likelihood (ML) estimation algorithm of the PHYLIP computer program. In this embodiment, HA gene segment that is to encode hemagglutinin (a membrane glycoprotein) is used as the selected gene segment and the corresponding gene segment.

Inputs of the first phylogenetic algorithm include the at least one selected gene segment of the gene sequence of the target biological virus and the corresponding at least one gene segment of the gene sequence of each of the related biological viruses in the group, i.e., the HA gene segment of the targeted biological virus H7N9 and the HA gene segments of the thirteen related biological viruses found in step 101.

In this embodiment, inputs of the second phylogenetic algorithm differ from those of the first phylogenetic algorithm. In detail, inputs of the second phylogenetic algorithm include, after undergoing length equalization processing to obtain equal sequence lengths, the at least one selected gene segment of the gene sequence of the target biological virus and the corresponding at least one gene segment of the gene sequence of each of the related biological viruses in the group, i.e., the HA gene segment of the targeted biological virus H7N9 and the HA gene segments of the related biological viruses in the group after undergoing length equalization processing. Since there is less restriction on the input data of UPMGA, it is suitable for analysis by approximation. On the other hand, the second phylogenetic algorithm (ML estimation algorithm) that requires length equalization processing is used for its calculation preciseness albeit using more calculation time. Based on the HA gene segment of the targeted biological virus H7N9 and the HA gene segments of the related biological viruses found in step 101, the length equalization processing is conducted using a sequence alignment algorithm to produce fourteen gene sequences (H7N9 and the thirteen related biological viruses found in step 101) of the same sequence length, which are the inputs of the second phylogenetic algorithm. In this embodiment, the sequence alignment algorithm is the Needleman-Wunsch algorithm.

The first phylogenetic tree in FIG. 2 includes a target external node corresponding to the HA gene segment of the targeted biological virus H7N9 , and a plurality of external nodes respectively corresponding to the HA gene segments of the related biological viruses. Similarly, the second phylogenetic tree in FIG. 3 includes a target external node corresponding to the HA gene segment of the targeted biological virus H7N9 , and a plurality of external nodes respectively corresponding to the HA gene segments of the related biological viruses.

In step 103, a computer determines a first subset of to-be-matched biological viruses from the first phylogenetic tree (first result), and the first subset includes the target biological virus, i.e., H7N9. The first subset of the to-be-matched biological viruses is determined based on node distances of the target external node of the target biological virus to the external nodes of the related biological viruses in the first subset. Similarly, the computer determines a second subset of the to-be-matched biological viruses from the second phylogenetic tree (second result), and the second subset includes the target biological virus H7N9. The second subset of the to-be-matched biological viruses is determined based on node distances of the target external node of the target biological virus to the external nodes of the related biological viruses in the second subset.

Referring to FIG. 2, H7N7 HA, H7N1 HA, H7N3 HA, and H7N2 HA are within a certain distance from the HA gene segment of the target biological virus H7N9. Therefore, the first subset of the to-be-matched biological viruses includes H7N9 HA, H7N7 HA, H7N1 HA, H7N3 HA, and H7N2 HA. Referring to FIG. 3, H7N7 HA, H7N3 HA, H7N2 HA, and H7N1 HA are within a certain distance from the HA gene segment of the target biological virus H7N9. Therefore, the second subset of the to-be-matched biological viruses includes H7N9 HA, H7N7 HA, H7N3 HA, H7N2 HA, and H7N1 HA.

In step 104, a computer determines a set of to-be-matched biological viruses from the first subset of the to-be-matched biological viruses and the second subset of the to-be-matched biological viruses. In this embodiment, the set of to-be-matched biological viruses is an intersection set of the first subset of the to-be-matched biological viruses and the second subset of the to-be-matched biological viruses. In this embodiment, since the first subset of the to-be-matched biological viruses and the second subset of the to-be-matched biological viruses happen to be identical, the set of to-be-matched biological viruses is H7N9, H7N7, H7N1, H7N3, and H7N2. It should be noted that, in other embodiments, the set of to-be-matched biological viruses may be a union set of the first subset of the to-be-matched biological viruses and the second subset of the to-be-matched biological viruses.

In step 105, a computer matches the full-length gene sequences of the to-be-matched biological viruses in the set of to-be-matched biological viruses, so as to find the loci-of-interest in the gene sequence of the target biological virus H7N9, in which the nucleotide at the loci-of-interest of the target biological virus H7N9 is different from that at a corresponding genomic locus of at least one of the other biological viruses in the set of to-be-matched biological virus. In this step, the matching is conducted using the Needleman-Wunsch algorithm operating on the set of to-be-matched biological viruses (i.e., H7N9, H7N7, H7N1, H7N3, and H7N2), and the following 523 loci-of-interest are found: 15, 17, 24, 25, 29, 31, 34, 35, 36, 37, 38, 39, 40, 41, 42, 43, 44, 46, 48, 50, 52, 53, 54, 55, 56, 57, 60, 62, 69, 72, 75, 84, 90, 94, 99, 102, 105, 111, 123, 126, 128, 129, 132, 133, 135, 141, 144, 147, 150, 153, 159, 165, 166, 167, 168, 169, 171, 172, 174, 175, 177, 178, 179, 180, 182, 183, 186, 189, 190, 192, 193, 194, 195, 204, 205, 207, 208, 209, 210, 213, 216, 219, 221, 225, 228, 234, 237, 238, 240, 241, 243, 245, 246, 249, 252, 261, 264, 270, 273, 276, 280, 281, 282, 283, 285, 289, 291, 294, 300, 301, 303, 304, 306, 314, 315, 321, 324, 327, 330, 333, 335, 336, 340, 341, 342, 352, 354, 355, 363, 366, 369, 370, 372, 373, 374, 375, 378, 381, 384, 389, 390, 397, 405, 411, 414, 417, 420, 429, 435, 438, 439, 441, 447, 450, 452, 453, 457, 462, 468, 477, 486, 490, 492, 495, 498, 501, 502, 507, 511, 513, 516, 519, 522, 525, 531, 535, 537, 540, 542, 546, 547, 549, 552, 554, 555, 556, 558, 564, 571, 573, 580, 582, 585, 588, 591, 593, 597, 598, 599, 600, 602, 603, 606, 609, 615, 618, 621, 624, 630, 632, 636, 637, 639, 648, 649, 651, 654, 657, 660, 666, 669, 672, 675, 676, 677, 678, 681, 684, 687, 690, 693, 694, 696, 697, 698, 699, 700, 701, 702, 703, 704, 705, 706, 707, 708, 709, 710, 711, 712, 713, 714, 715, 716, 717, 718, 719, 720, 721, 723, 726, 729, 732, 735, 741, 742, 744, 747, 748, 750, 756, 765, 768, 773, 786, 798, 799, 802, 804, 810, 813, 814, 816, 819, 822, 823, 825, 828, 829, 831, 835, 837, 845, 846, 849, 851, 852, 853, 855, 859, 861, 862, 863, 868, 869, 870, 873, 874, 881, 882, 885, 891, 894, 897, 901, 903, 906, 907, 908, 909, 910, 912, 915, 918, 921, 927, 930, 931, 932, 933, 936, 937, 939, 942, 945, 951, 954, 955, 957, 963, 966, 969, 970, 971, 972, 975, 978, 979, 981, 982, 987, 993, 998, 999, 1002, 1005, 1008, 1011, 1012, 1013, 1014, 1017, 1021, 1022, 1023, 1029, 1032, 1038, 1041, 1044, 1047, 1050, 1056, 1059, 1062, 1071, 1077, 1080, 1081, 1083, 1086, 1095, 1101, 1113, 1119, 1120, 1122, 1131, 1134, 1137, 1149, 1152, 1155, 1161, 1167, 1170, 1176, 1182, 1185, 1188, 1191, 1195, 1196, 1197, 1203, 1206, 1209, 1212, 1218, 1219, 1221, 1230, 1233, 1238, 1242, 1243, 1245, 1249, 1251, 1257, 1260, 1263, 1266, 1269, 1272, 1278, 1279, 1284, 1285, 1287, 1293, 1296, 1297, 1299, 1305, 1308, 1311, 1317, 1318, 1320, 1321, 1324, 1326, 1335, 1341, 1344, 1350, 1356, 1359, 1373, 1380, 1383, 1386, 1389, 1392, 1394, 1395, 1397, 1398, 1404, 1407, 1410, 1413, 1422, 1428, 1434, 1437, 1440, 1443, 1449, 1452, 1455, 1464, 1465, 1467, 1470, 1475, 1476, 1479, 1482, 1485, 1494, 1500, 1503, 1505, 1506, 1507, 1515, 1516, 1517, 1518, 1522, 1524, 1525, 1530, 1545, 1551, 1554, 1558, 1560, 1563, 1566, 1569, 1572, 1578, 1579, 1581, 1584, 1585, 1587, 1602, 1614, 1615, 1617, 1623, 1638, 1639, 1641, 1644, 1650, 1653, 1654, 1656, 1673.

As compared to performing biochemistry experiments on the 1706 loci in the HA gene segment, only 523 (30.66%) of the 1706 loci in the HA gene segment are required for performing experiments on, significantly reducing experiment costs and time.

It is worth mentioning that instead of the HA gene segment, the NA gene segment may be used as the selected gene segment, and similar results may be obtained.

While this embodiment was illustrated using the influenza A virus H7N9, the present invention is not limited in this respect. The technique of this invention may also be applied to other biological viruses, such as the Enterovirus. Moreover, if a genus or a family of biological viruses to which the target biological virus belongs is not large, step 101 may be omitted, and the entire genus or family of biological viruses to which the target biological virus belongs may serve as the group of related biological viruses in step 102. In other embodiments, if the gene sequence of the target biological virus has yet to be defined with corresponding gene segments, the selected gene segment in step 102 may be the full-length gene sequence of the target biological virus. Furthermore, while the phylogenetic tree information is generated by executing two different phylogenetic algorithms in this embodiment, one or more than two phylogenetic algorithms may be utilized to generate the phylogenetic tree information in other embodiments of this invention.

In summary, at least one phylogenetic algorithm is executed to generate phylogenetic tree information, from which a set of to-be-matched biological viruses may be found. Thereafter, full-length gene sequences of the to-be-matched biological viruses in the set are matched, so as to, find loci-of-interest in the gene sequence of a target biological virus. By such virtue, the scope of biochemistry experiments performed to find immune genomic loci that may involve immunogenicity is significantly reduced.

While the present invention has been described in connection with what is considered the most practical embodiment, it is understood that this invention is not limited to the disclosed embodiment but is intended to cover various arrangements included within the spirit and scope of the broadest interpretation so as to encompass all such modifications and equivalent arrangements.

1. A computer-implemented method for quick-search of loci-of-interest in a gene sequence of a target biological virus, the computer-implemented method comprising:
A) finding a set of to-be-matched biological viruses from a group of related biological viruses that are related to the target biological virus, including
a1) generating phylogenetic tree information using at least one computer that executes at least one phylogenetic algorithm, the phylogenetic tree information being generated based on at least one selected gene segment of the gene sequence of the target biological virus and a corresponding at least one gene segment of a gene sequence of each of the related biological viruses in the group, the corresponding at least one gene segment corresponding to the at least one selected gene segment, and
a2) determining the set of to-be-matched biological viruses based on the phylogenetic tree information thus generated; and
B) matching, using a computer, the gene sequences of the to-be-matched biological viruses in the set so as to find the loci-of-interest in the gene sequence of the target biological virus.

2. The computer-implemented method of claim 1, wherein the loci-of-interest are possible immune genomic loci that may involve immunogenicity.

3. The computer-implement method of claim 1, wherein the loci-of-interest are associated with mutation loci among the gene sequences of the to-be-matched biological viruses in the set.

4. The computer-implemented method of claim 1, wherein, in sub-step a1), the phylogenetic tree information is generated by executing a first phylogenetic algorithm to obtain a first result, and by executing a second phylogenetic algorithm to obtain a second result, and in sub-step a2), the set of to-be-matched biological viruses is determined s based on the first result and the second result.

5. The computer-implemented method of claim 4, wherein sub-step a2) includes:
determining a first subset of the to-be-matched biological viruses from the first result, the first subset including the target biological virus;
determining a second subset of the to-be-matched biological viruses from the second s result, the second subset including the target biological virus; and
determining the set of to-be-matched biological viruses from the first subset and the second subset.

6. The computer-implemented method of claim 5, wherein the set of to-be-matched biological viruses is determined based on node distances of the target biological virus to the related biological viruses in the first subset and the second subset.

7. The computer-implemented method of claim 4, wherein the set of to-be-matched biological viruses is determined according to node distances of the target biological virus to the related biological viruses based on the first result and the second result.

8. The computer-implemented method of claim 4, wherein:
inputs of the first phylogenetic algorithm include the at least one selected gene segment of the gene sequence of the target biological virus and the corresponding at least one gene segment of the gene sequence of each of the related biological viruses in the group, and
inputs of the second phylogenetic algorithm include, after undergoing length equalization processing to obtain equal sequence lengths, the at least one selected gene segment of the gene sequence of the target biological virus and the corresponding at least one gene segment of the gene sequence of each of the related biological viruses in the group.

9. The computer-implemented method of claim 8, wherein the length equalization processing is conducted using a sequence alignment algorithm.

10. The computer-implemented method of claim 9, wherein the sequence alignment algorithm is the Needleman-Wunsch algorithm.

11. The computer-implemented method of claim 8, wherein the first phylogenetic algorithm is the Unweighted Pair Group Method with Arithmetic Mean (UPGMA) algorithm and the second phylogenetic algorithm is the maximum-likelihood estimation algorithm.

12. The computer-implemented method of claim 4, wherein the first phylogenetic algorithm is the Unweighted Pair Group Method with Arithmetic Mean (UPGMA) algorithm.

13. The computer-implemented method of claim 4, wherein the second phylogenetic algorithm is the maximum-likelihood estimation algorithm.

14. The computer-implemented method of claim 1, wherein the phylogenetic algorithm executed in step A) includes at least one of the Unweighted Pair Group Method with Arithmetic Mean (UPGMA) algorithm and the maximum-likelihood estimation algorithm.

15. The computer-implemented method of claim 1, further comprising, prior to step A):
0) using a computer that executes a clustering algorithm to find the group of related biological viruses from a genus of a family of biological viruses to which the target biological virus belongs, the clustering algorithm operating based on at least one selected gene segment of the gene sequence of the target biological virus and a corresponding at least one gene segment of a gene sequence of each of the biological viruses in the genus.

16. The computer-implemented method of claim 15, wherein the target biological virus belongs to the Influenza A virus genus, and in step Q), the selected gene segment of the gene sequence of the target biological virus operated upon by the clustering algorithm is PB2 gene segment that encodes PB2 RNA polymerase.

17. The computer-implemented method of claim 15, wherein the clustering algorithm is the Unweighted Pair Group Method with Arithmetic Mean (UPGMA) algorithm.

18. The computer-implemented method of claim 1, wherein the target biological virus belongs to the Influenza A virus genus, and in step A), the selected gene segment of the gene sequence of the target biological virus based on which the phylogenetic tree information is generated is HA gene segment that encodes hemagglutinin (HA).

19. The computer-implemented method of claim 1, wherein the target biological virus belongs to the Influenza A virus genus, and in step A), the selected gene segment of the gene sequence of the target biological virus based on which the phylogenetic tree information is generated is NA gene segment that encodes neuraminidase (NA).

20. The computer-implemented method of claim 1, wherein the matching in step B) is conducted using the Needleman-Wunsch algorithm.
